(19) United States
(12) Patent Application Publication
(10) Pub. No.: US 2025/0140425 A1
(43) Pub. Date: May 1, 2025

(54) SYSTEM AND METHOD FOR FACILITATING ENGAGEMENT OF ALLIED HEALTH ADVISORS

(71) Applicant: Matthew DREWETT, Munno Para West (AU)

(72) Inventor: Matthew DREWETT, Munno Para West (AU)

(21) Appl. No.: 18/835,685

(22) PCT Filed: Jan. 31, 2023
(86) PCT No.: PCT/AU2023/050054

§ 371 Date: Aug. 2, 2024

(30) Foreign Application Priority Data
Feb. 2, 2022 (AU) 2022900193

(51) Int. Cl. G16H 80/00; G06Q 30/0601; G16H 20/30; G16H 20/60; G16H 40/20

(52) U.S. Cl. CPC G16H 80/00; G06Q 30/0639; G16H 20/30; G16H 20/60; G16H 40/20

(57) ABSTRACT

Facilitation of engaging health advisor(s) include: receiving data of consumer(s) seeking to engage health service provider(s); storing in a database including a data structure, data relating to health service provider(s) offering consumer services (the data including a region where the health service provider offers services), and the health service provider qualification(s); receiving from a consumer a request that includes: a health-related service sought by the consumer, a location where the consumer seeks to receive services, and consumer requirement(s) relating to the service; retrieving data of the requesting consumer; generating, based upon the request and the retrieved consumer data, a query for utilizing the data structure to identify provider(s) who offer services within a region where the location resides and having minimum qualifications required to provide the service sought and satisfying additional consumer requirement(s); and providing to a consumer device, a result of the search conducted based upon the search query.

### FIELD OF THE INVENTION

The present invention relates to a system and method for assisting consumers to engage allied health advisors, such as personal trainers, fitness trainers and weight loss advisors. In particular, the system and method provides a digital platform for such advisors to offer and provide their services, and for consumers seeking such services to locate and engage providers that meet their specific requirements.

### BACKGROUND OF THE INVENTION

The allied health industry includes a broad range of health professionals who are not registered medical practitioners but who specialise in health-related services, e.g. personal trainers, fitness trainers, weight loss advisors, and the like. Despite the significant health benefits that arise from engaging such service providers, many consumers are reluctant to engage allied health providers due to factors such as having insufficient time and cost. For example, consumers seeking to engage a personal trainer or similarly qualified individual may be reluctant due to the relatively high cost of personal trainers who typically provide their services at a gym or fitness centre.

The typical arrangements between personal trainers and their clients contributes to the relatively high cost of a personal trainer, since it is difficult for personal trainers to provide services to clients without the support of (or association with) a facility such as a gym or fitness centre who impose a fee upon the individual offering services from their facility. However, this increases the cost of the service to the client since the personal trainer will typically pass on the fee as a disbursement to the client.

It is also difficult for personal trainers and other allied health providers to offer their services when clients are unable to ascertain the qualifications and/or experience of the service provider. As a result, consumers regularly rely upon a gym or fitness centre to act as a “qualification agent” based upon their expectation that the gym or fitness centre will only allow accredited or appropriately qualified personal trainers with a good reputation to offer services at their facility.

Accordingly, various factors contribute to the relatively high cost and difficulty associated with consumers engaging the services of allied health advisors and hence, an alternative arrangement is required to reduce the relatively high cost of providing such services whilst also satisfying the requirements of consumers with respect to locating and engaging a provider with appropriate skill, experience and reputation regarding the services they provide.

There is also a wastage of computing and memory resources associated with the conventional process of engaging allied health providers, since consumers need to spend a considerable amount of time utilising their devices (e.g. smart phones or tablets) to research the location and the experience and/or qualifications of providers, to initiate contact with providers, and to maintain an ongoing engagement. This has the effect of consuming substantial data communications bandwidth and hence causes significant computer and network resource usage. Inefficient use of the available bandwidth of data communications networks is clearly undesirable since slow and/or unreliable data communications are frustrating (and expensive) for users. This problem is exacerbated during times of high network traffic which consume available bandwidth.

The computer-implemented system and method of the present invention seeks to address the above identified problems or at least provide an alternative solution to same.

The reference to any prior art in this specification is not, and should not be taken as, an acknowledgement or any suggestion, that the prior art forms part of the common general knowledge.

### SUMMARY OF THE INVENTION

In one aspect, the present invention provides a computer-implemented method for facilitating the engagement of allied health advisors, the method including receiving, by one or more processors, data relating to a plurality of consumers seeking to engage one or more allied health service providers, storing, by one or more processors, in a database including a data structure, data relating to a plurality of allied health service providers offering their services to consumers, the data including a geographical region in which the allied health service provider offers their services, and one or more qualifications of the allied health service provider, receiving, by one or more processors, a request from a consumer of the plurality of consumers that seek to identify, by interrogating that data structure, a subset of the plurality of the allied health service providers whose stored data satisfied the consumer request for an allied health service provider, the request including at least a health-related service sought by the consumer, a location at which the consumer seeks to receive the health-related service, and one or more requirements of the consumer in relation to the health-related service, including one or more of a cost associated with the service, a characteristic of the service, or an availability of the consumer, retrieving, by one or more processors, data relating to the consumer to which the request relates from the data relating to the plurality of consumers seeking to engage one or more allied health service providers, generating and performing, by one or more processors, based upon the request received and the retrieved data relating to the consumer, a query of the data structure to identify a subset of the plurality of allied health service providers who offer services within a geographical region in which the location resides, have minimum qualifications required to provide the health-related service sought by the consumer, and satisfy the one or more additional requirements of the consumer, wherein the query includes an exact keyword match query and a minimum similarly threshold analysis such that, when the exact keyword match query generates no relevant search results, the similarly threshold analysis is performed to generate results solely according to a comparison between the query and the data relating to the plurality of allied health service providers satisfying a minimum similarly threshold, and providing, by one or more processors, to a data communications device associated with the consumer, a result of the search conducted according to the exact keyword match search query and/or the minimum similarity threshold analysis.

Accordingly, only those allied health service providers who have minimum qualifications to provide the health-related service sought by the consumer and satisfy the one or more requirements of the consumer, and who offer services in a geographical region that includes the location specified by the consumer, are identified in response to the search query.

In an embodiment, the method further includes generating, by one or more processors, a response to the search query, the response identifying the subset of allied health service providers from the plurality of allied health service providers, and causing, by one or more processors, the data communications device associated with the particular consumer to display the response.

In an embodiment, the subset of allied health service providers included in the search query response may be sorted (ordered) according to one or more parameters specified (selected) by the particular consumer, or may be sorted (ordered) automatically according to pre-defined parameters.

In an embodiment, the method further includes receiving, by one or more processors, from the data communications device associated with the consumer, a selection of a particular allied health service provider from the subset of allied health service providers.

In an embodiment, when the one or more requirements of the consumer in relation to the health-related service relates to a cost associated with the service, the consumer may specify a maximum hourly rate that the consumer is prepared to pay, when the one or more requirements relates to a characteristic of the service (eg. the consumer may prefer online or in-person service), and when the one or more requirements relates to an availability of the consumer, the consumer may only be available to receive the service on weekends.

In an embodiment, the stored data relating to the allied health service providers includes details relating to the cost of services provided by the allied health service provider, the characteristics of services offered by the allied health service provider, and the availability of the allied health service provider, and when the one or more requirements specified in the user request specifies a particular cost of the service provider, characteristic of the service provider, and/or availability of the consumer, a determination regarding whether the additional requirements are satisfied will involve comparing the one or more requirements against the stored data relating to the allied health service providers.

In an embodiment, the allied health service providers provide health-related services including one or more of personal training, fitness training, diet, nutrition, weight loss, and/or movement/mobility.

In an embodiment, the one or more qualifications of the allied health service provider includes one or more of an academic qualification, a technical qualification, experience (eg. number of years of relevant experience), an accreditation, consumer rating, and/or reputation.

In an embodiment, the data communications device associated with the particular consumer operates a software application that enables the consumer to prepare and submit the request, to view the response to the request, and to select a particular allied health service provider from those listed in the search query response.

In an embodiment, details relating to each allied health service provider are received via a data communications device that also operates a software application, the software application enabling each allied health service provider to submit details relating to the geographical region in which the allied health service provider offers their services, the qualifications of the allied health service provider, and any additional relevant details regarding the services they provide, prior to storage and indexing of those details in the data structure.

In an embodiment, the software application enables the allied health service providers to publish material for viewing by consumers, including public materials viewable by all consumers, and private materials viewable only by those consumers who have engaged the services of the allied health service provider.

In an embodiment, the published materials include one or more of instructional programs created by the allied health service provider (eg. online programs relating to fitness, weight loss, diet, etc that consumers may access virtually rather than in person), and a schedule of the allied health service provider.

In an embodiment, the published schedule of the allied health service provider may be viewed by consumers and the schedule may be displayed on the data communications device of each consumer in a configuration enabling each consumer to view available appointment times of the allied health service provider and to schedule an appointment during one of the available times.

In an embodiment, when a particular allied health service provider has been selected by the particular consumer and the engagement of the particular allied health provider confirmed, the software application further provides a payment gateway enabling consumers to pay for services provided by the allied health provider including payment for access to programs and in respect of appointments scheduled with the allied health service provider.

In an embodiment, when a particular allied health service provider has been selected by a consumer and the engagement of the allied health service provider has been confirmed, the software application further provides a communication gateway enabling the consumer and health service provider to exchange messages and other materials, engage in video conferencing and schedule appointments such that health service providers may arrange the provision of online or in-person services at an agreed time and location for an agreed cost.

In an embodiment, the software application further enables each of the consumers and the allied health service providers to monitor and update any submitted details, including any additional details submitted for the purpose of facilitating the identification of the consumer and/or allied health service provider.

In an embodiment, the data communications device associated with the consumer and/or allied health service provider is a mobile device such as a tablet or smart phone.

In another aspect, the present invention provides a system for facilitating the engagement of allied health service providers, the system including one or more processors operable to receive data relating to a plurality of consumers seeking to engage one or more allied health service providers, store, in a database including a data structure, data relating to a plurality of allied health service providers offering their services to consumers, the data including a geographical region in which the allied health service provider offers their services, and one or more qualifications of the allied health service provider, receive a request from a consumer of the plurality of consumers that seek to identify, by interrogating that data structure, a subset of the plurality of the allied health service providers whose stored data satisfied the consumer request for an allied health service provider, the request including at least a health-related service sought by the consumer, a location at which the consumer seeks to receive the health-related service, and one or more requirements of the consumer in relation to the health-related service including one or more of a cost associated with the service, a characteristic of the service, or an availability of the consumer, retrieve data relating to the consumer to which the request relates from the data relating to the plurality of consumers seeking to engage one or more allied health service providers, generate and perform, based upon the request received and the retrieved data relating to the consumer, a query of the data structure to identify a subset of the plurality of allied health service providers who offer services within a geographical region in which the location resides have minimum qualifications required to provide the health-related service sought by the consumer, and satisfy the one or more additional requirements of the consumer wherein the query includes an exact keyword match query and a minimum similarly threshold analysis such that, when the exact keyword match query generates no relevant search results, the similarly threshold analysis is performed to generate results solely according to a comparison between the query and the data relating to the plurality of allied health service providers satisfying a minimum similarly threshold; and provide, to a data communications device associated with the consumer, a result of the search conducted according to the exact keyword match search query or the minimum similarly threshold analysis.

In a further aspect, the present invention provides non-transitory computer-readable medium having a plurality of computer instructions executable by one or more processors that, when executed, cause the one or more processors to: receive data relating to a plurality of consumers seeking to engage one or more allied health service providers, store, in a database including a data structure, data relating to a plurality of allied health service providers offering their services to consumers, the data including a geographical region in which the allied health service provider offers their services, and one or more qualifications of the allied health service provider, receive a request from a consumer of the plurality of consumers that seek to identify, by interrogating that data structure, a subset of the plurality of the allied health service providers whose stored data satisfied the consumer request for an allied health service provider, the request including at least a health-related service sought by the consumer, a location at which the consumer seeks to receive the health-related service, and one or more requirements of the consumer in relation to the health-related service including one or more of a cost associated with the service, a characteristic of the service, or an availability of the consumer; retrieve data relating to the consumer to which the request relates from the data relating to the plurality of consumers seeking to engage one or more allied health service providers; generate and perform, based upon the request received and the retrieved data relating to the consumer, a query of the data structure to identify a subset of the plurality of allied health service providers who offer services within a geographical region in which the location resides have minimum qualifications required to provide the health-related service sought by the consumer, and satisfy the one or more additional requirements of the consumer wherein the query includes an exact keyword match query and a minimum similarly threshold analysis such that, where the exact keyword match query generates no relevant search results, the similarly threshold analysis is performed to generate results solely according to a comparison between the query and the data relating to the plurality of allied health service providers satisfying a minimum similarly threshold; and provide, to a data communications device associated with the consumer, the results of the search conducted according to the exact keyword match and/or the minimum similarly threshold analysis search query.

### BRIEF DESCRIPTION OF THE DRAWINGS

Features of the present disclosure are illustrated by way of example and not limited in the following Figure(s), in which like numerals indicate like elements, in which:

FIG. 1 provides an overview of a system according to an embodiment of the present invention showing, in particular, the interaction between various system components;

FIG. 2 illustrates an exemplary diagram illustrating a server component associated with the system illustrated in FIG. 1;

FIG. 3 illustrates an exemplary flow diagram of a process that enables an allied health service provider to use a software application to advertise and promote their services to potential clients including to create and maintain an advisor profile;

FIG. 4 illustrates an exemplary flow diagram of a process that enables a consumer to download and install a software application, and subsequently access, or register to use, the software application, for interaction with the system illustrated in FIG. 1;

FIG. 5 illustrates an exemplary flow diagram of process that enables the consumer to search for, locate and engage allied health service providers to address their health requirements;

FIG. 6 illustrates an allied health service provider delivering a service or program to a particular consumer; and

FIG. 7 illustrates an exemplary flow diagram of a process that enables the allied health service provider to be paid for their services via the software application.

### DETAILED DESCRIPTION OF EMBODIMENT(S) OF THE INVENTION

For simplicity and illustrative purposes, the present disclosure is described by referring to embodiment(s) thereof. In the following description, numerous specific details are set forth to provide a better understanding of the present disclosure. It will be readily apparent, however, that the current disclosure may be practiced without limitation to the specific details. In other instances, some methods and structures have not been described in detail to avoid obscuring the present disclosure.

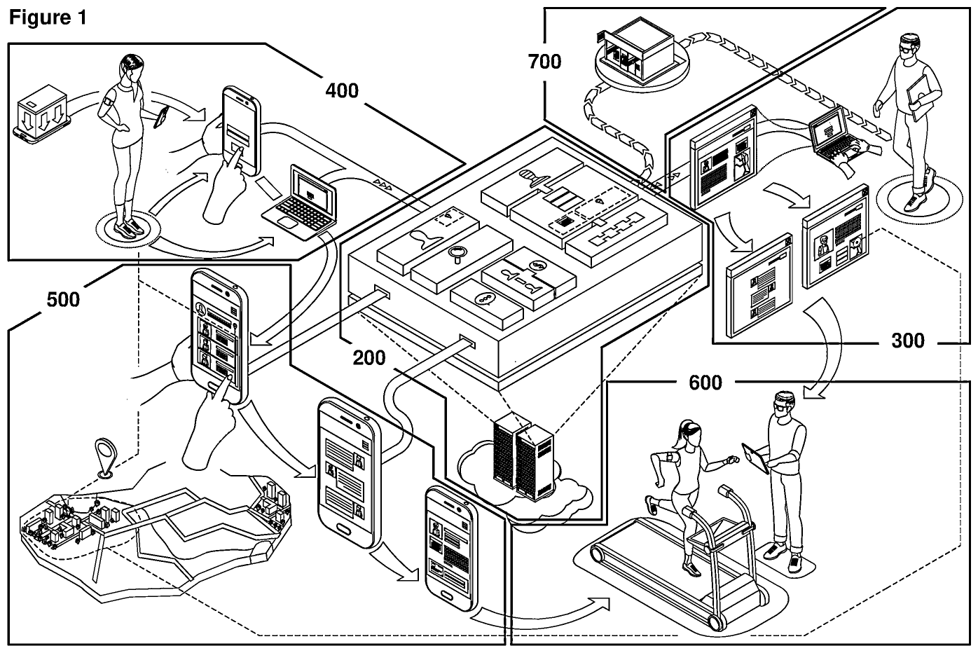

According to an embodiment, the present invention provides a system and method for facilitating the engagement, by consumers (30), of allied health advisors (also referred to herein as allied health service providers) (60), as depicted in FIG. 1. The system and method provides a platform that hosts computer-executable software applications (40/70), wherein the applications (40/70) are accessible by a plurality of users (30/60). For example, consumers (30) seeking the services of an allied health service provider (60) may access software application (40), and the allied health service provider (60) may access their own dedicated software application (70), although each application will provide similar functionality.

The platform enables the generation of search results (280), wherein the search results display a listing of available service providers (60) who meet certain criteria of a search conducted by a particular consumer (30). An estimate of the service provider's charges for providing particular services may also be displayed to the consumer (30), thereby enabling the consumer (30) to confirm selection of a service provider (60) from the search results (280) and attend to payment to the service provider (60). The particular criteria upon which certain service providers (60) are shortlisted in search results is described in greater detail below.

The central server (20) shown in FIG. 1 maintains one or more processors and/or databases for performing functions, including interfacing with data and communications devices (50) associated with consumers (30) as well as allied health service providers (60). For example, such communication involves the central server (20) receiving data from the consumer (30) regarding their requirements, including a request specifying a health-related service sought by the consumer (30), a location (270) at which the consumer prefers to receive the health-related service (320), and one or more requirements of the consumer (30) in relation to the health-related service.

The communication also involves the central server (20) receiving information from each allied health service provider (60) regarding a geographical region (also referred to herein as a service area (240)) in which the service provider (60) offers their services, and one or more qualifications of the service provider (60).

Once such data has been received from each consumer (30) and service provider (60), they may utilise their data communication devices (50) (which are operable to connect with the server (20) via a data communications network) to access the functionality of the respective software applications (40) and (70).

Accordingly, the server (20) is operable to process requests from consumers (30) to search available service providers (60), and in response, identify one or more allied health service providers (60) who offer services within a geographical region in which the location (270) of the consumer (30) resides. The consumer (30) can be confident that the identified service providers (60) will have the minimum qualification(s) required to provide the health-related service sought by the consumer (30), whilst satisfying the one or more requirements of the consumer (30). The server (20) may then cause the computing device (50) associated with the consumer (30) to display, substantially in real-time, a graphical representation of available allied health service providers (60) based upon those particular service providers (60) offering services within a geographical region in which the location (270) registered by the consumer (30) resides, having the minimum qualifications required to provide the health-related service sought by the consumer (30), and the ability to satisfy the one or more requirements of the consumer (30).

The person skilled in the relevant field of technology will appreciate that the platform provides a technical solution to various problems including how to enable consumers (30) to efficiently locate and engage with an allied health service provider (60) capable of satisfying their various requirements, and further, how to provide allied health service providers (60) with the ability to more efficiently promote their services and engage with clients. The result is an online marketplace for allied health service providers (60), such as personal trainers, fitness trainers and weight loss advisors, who can use the platform to advertise and promote their services to potential clients, and clients (30) who can use the platform to locate and engage service providers in their local area and interact directly with them to obtain the services they require. These advantages are achieved through the use of a data structure which facilitates the conservation of computer and network resource usage.

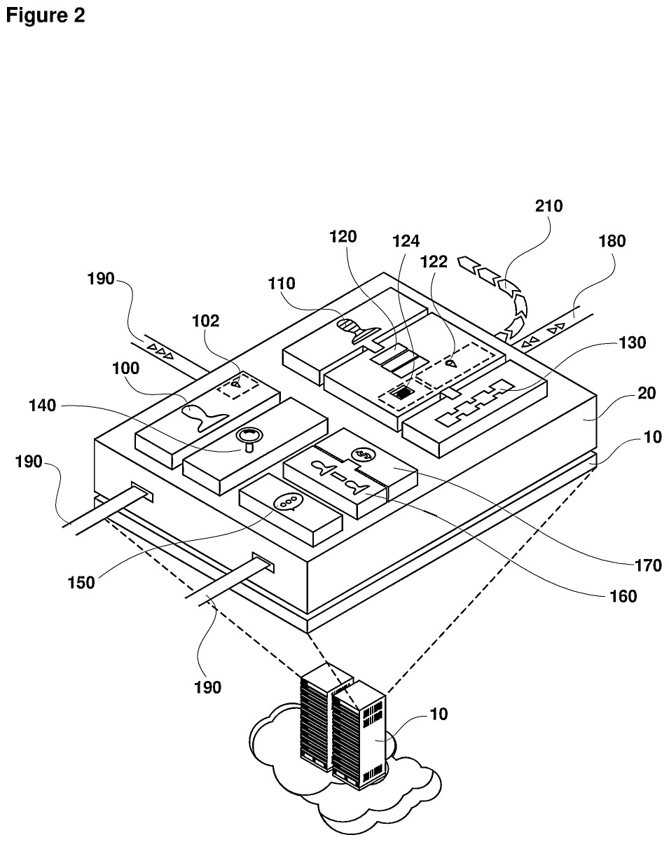

FIG. 1 is divided into segments which are further expanded in subsequent FIGS. 2 to 7. In particular, segment 200 of FIG. 1 shows the server component (20) with which a software application (40) operating on a data communications device (50) of a consumer (30) is configured to communicate. The server component is also operable to communicate with data communication devices (50) associated with allied health service providers (60) seeking to offer their services, and in particular with software applications (70). It will be apparent to the person skilled in the relevant field of technology that the software application (40/70) may be a mobile application (such as application 40A)) or a web application (such as web application (40B)), and similarly, the data communication device (50) utilised by each user may be a portable device such as a mobile phone (50A) or laptop (50B), or alternatively a fixed location device such as a personal computer (not shown). The server component (20) is additionally detailed in FIG. 2.

The skilled person will appreciate that the steps described herein may be executed by the devices (50), wherein such operations are facilitated by the software applications (40/70) operating on each device. According to another implementation of the present invention, the server (20) is programmed to provide all of the functions described herein where they cannot be provided locally on the user devices (50) or where it may be impractical to do so. In other words, the steps described herein as performed by the device (50), or components of the device (50) that are described as implementing the invention, may be associated with hardware that is located externally of the device (50), such as the remote central server (20) for example (i.e. in a distributed architecture). Different arrangements are possible in this regard, and alternate variations will be apparent to the person skilled in the relevant field of technology.

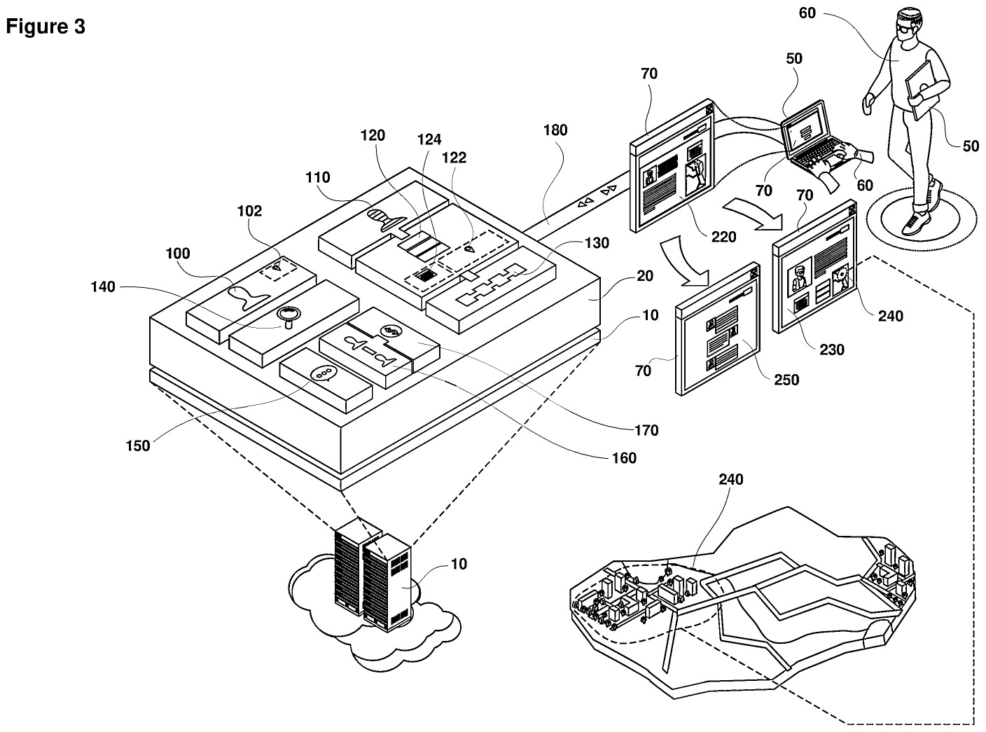

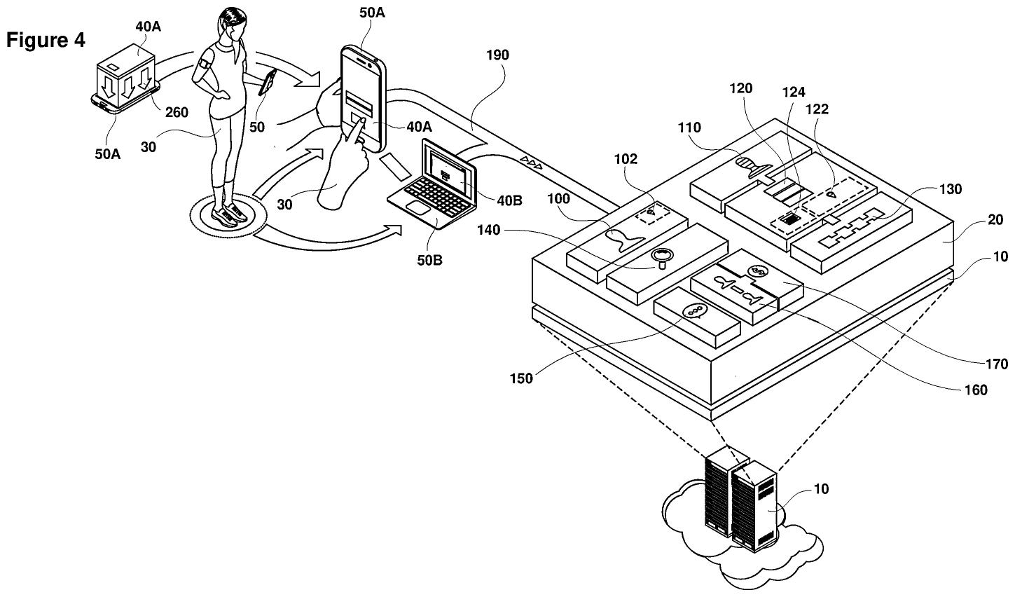

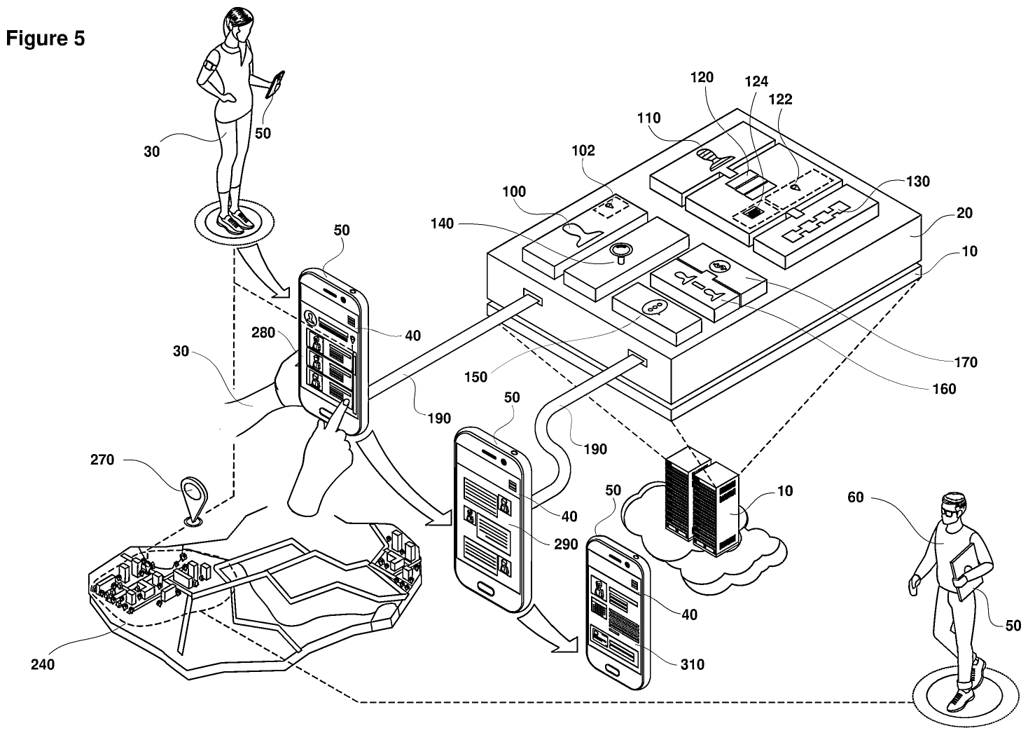

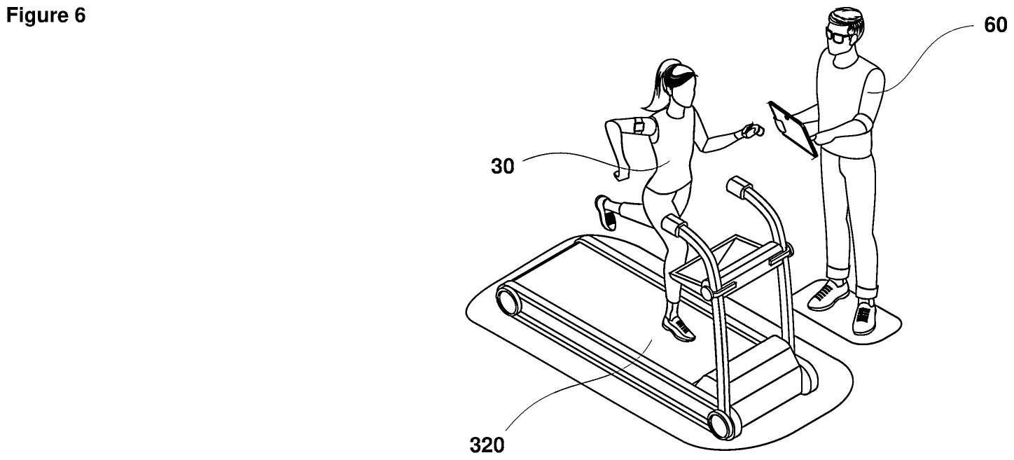

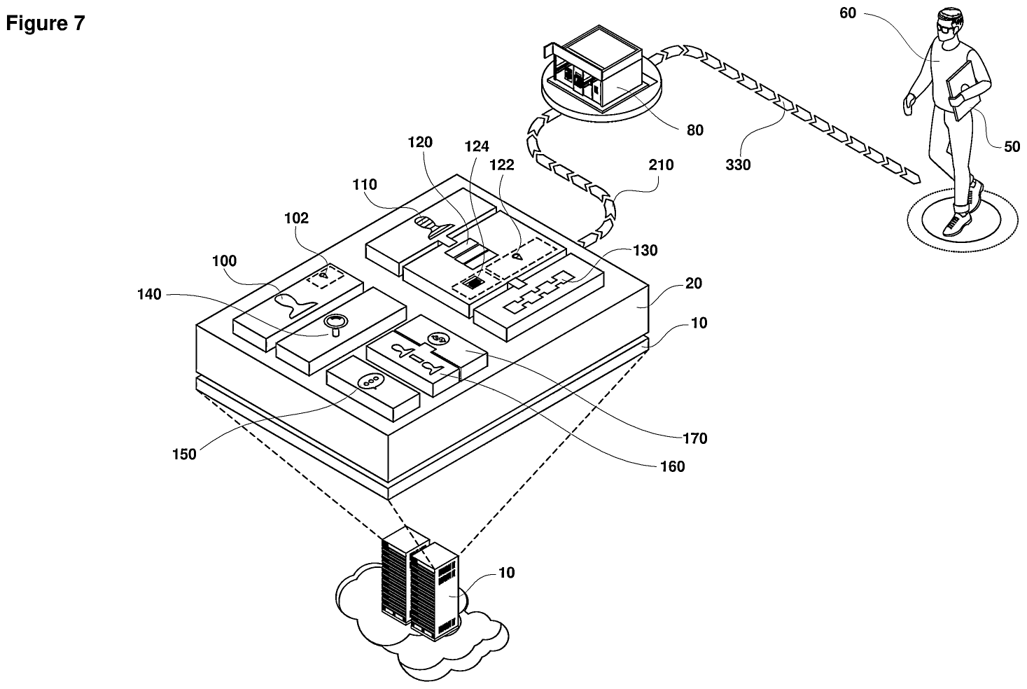

Segment 300 of FIG. 1 shows an allied health service provider (60) interacting with software application (70) to create a profile to advertise and promote their services to potential clients and to receive and respond to inquiries, as further detailed in FIG. 3. Segment 400 of FIG. 1 shows a consumer (30) downloading and installing the relevant application (40) to enable the consumer (30) to access the full functionality of the application (40), as described herein and further detailed in FIG. 4. Segment 500 of FIG. 1 illustrates the consumer (30) utilising the software application (40) to search for available service providers (60) who meet certain criteria according to a minimum similarity threshold, and to subsequently select a service provider for engagement, as further detailed in FIG. 5. Segment 600 of FIG. 1 illustrates the provision of a particular service (320) to a consumer (30) by service provider (60) following engagement of the service provider (60), as further detailed in FIG. 6. Finally, segment 700 of FIG. 1 illustrates a method by which the service provider (60) may be provided payment (i.e. via a financial institution (80) associated with the service provider (60)) in respect of their services, as detailed in FIG. 7.

As mentioned above, FIG. 2 shows in greater detail segment 200 of FIG. 1. In particular, FIG. 2 shows the server component (20) which may include infrastructure (10) upon which the platform of the present invention operates. The infrastructure (10) may be local or cloud-based.

The central server (20) may operate one or more processors and maintain one or more databases to enable the following functionality and/or storage:
Consumer database (100) storing details relating to each registered consumer (30) (eg. name, address, contact details etc.), as well as any additional data which may be relevant for the purpose of identifying each consumer (30). Where possible, these details are verified using identification verification services;
Consumer location record (102) storing details relating to a location (270) registered by the individual consumers (30). The location may be a physical location of the consumer (30) (eg. the user's home address), a current location of the consumer (30), or a preferred location specified by the user (30);
Allied health service provider database (110) storing details relating to each registered service provider (60) (eg. name, address, contact details, business details, etc.) as well as any additional data which may be relevant for the purpose of identifying each service provider (60). Again, where possible, these details may be verified using identification verification services;
Service provider profile database (120) storing profiles created by each registered allied health service provider (60);
Service provider geographical service area record (122) storing details relating to the specific geographical area(s) serviced by each provider (60) including for example service area (240) shown in FIG. 5, and details of which may be included in the particular service provider's profile stored in database (120);
Service provider details database (124) storing additional details registered by each service provider (60) including their qualifications and the services they offer to consumers (30), details relating to costs associated with particular services, characteristics of particular services, and an availability of the service provider (60) (e.g. the advisor's scheduled hours of operation);
Service provider services and programs record (130) storing specific details relating to the programs offered by each registered service provider (60) including online programs and schedules that may be shared with clients and potential clients;
Consumer search functionality (140) which may utilise a machine learning algorithm to enable relevant search results to be generated based upon a request received from a consumer (30) to search for allied health service providers (60) offering particular health services in their local area and in particular at their specified location (270);
Enquiry database (150) storing details relating to particular enquiries received from consumers (30), including general enquiries regarding health programs and services provided by service providers (60);
Bookings database (160) storing details relating to bookings made by consumers (30) with particular service providers (60) after searching for and subsequently selecting a service provider (60) to provide health-related services. For example, database (160) may store details relating to specific times and dates for the delivery of services as agreed between a consumer (30) and a service provider (60); and
Payment gateway (170) for managing financial transactions including allowing for funds to be transferred from consumers (30) to allied health service providers (60) for the delivery of health-related services by the service provider (60), (eg. to a financial institution (80) associated with the service provider (60)).

FIG. 2 also depicts server (20) configured to enable communication (180) with device (50) associated with each allied health service provider (60), and further, how server (20) is configured to communicate (190) with the consumer devices (150). Also shown in FIG. 2 is the server's ability to communicate (210) with a financial institution (80) associated with each allied health service provider (60). The communications described above may occur directly, or via the internet or a similar network.

FIG. 3 shows in greater detail Segment 300 of FIG. 1, and in particular, an example interface (220) associated with the service provider's software application (70) which enables a registered service provider (60) to create, view and maintain their profile (120), as well as accept bookings and interact with clients (30).

Also shown in FIG. 3 is an example interface (230) associated with software application (70) which enables service providers (60) to edit their profile including details relating to themselves, their qualifications, the services they offer to consumers (30), the geographic area (240) where services are able to be provided, as well as any additional detail relating to the cost of their services, characteristics of their services and their availability. Each time service provider details are added or edited, the relevant database (110, 120, 122, 124, 130) is automatically updated. Additionally, the interface (230) may enable service providers to enter details relating to particular online programs and/or schedules which they may prefer to share with clients (including potential clients).

FIG. 3 also shows an interface (250) associated with software application (70) which allows allied health service providers (60) to receive and respond to online enquiries from customers (30) (including potential customers), regarding their services. In the particular embodiment shown, a live chat functionality is provided by interface (250).

The service provider (60) may specify in their profile a characteristic of a particular service offering which may include, for example, details relating to whether the service is provided online and/or in-person. In another example, the service provider (60) may specify their availability by identifying particular hours on weekdays and/or weekends on which the service provider (60) is prepared to work.

The one or more qualifications specified by the service provider (60) may include an academic qualification, a technical qualification, experience (e.g. number of years of relevant experience, and accreditation consumer rating, and/or reputation).

FIG. 4 shows in greater detail segment 400 of FIG. 1, and in particular, the steps associated with a consumer (30) installing (260) the software application (40) on their device (50), and subsequently accessing a user login and registration interface associated with the application (40). Such access may be granted after the consumer (30) has installed the application (40) which may be achieved by downloading the application (40) from an application store. Each consumer (30) may create an account using the application (40), and the account information may be stored in database (100). As described above, the database (100) may capture information sufficient to enable each consumer (30) to be correctly identified.

It will be appreciated that similar steps as those shown in FIG. 4 will apply equally to the installation of software application (70) upon a device (50) associated with the service provider (60). The server provider (600) may subsequently access a user login and registration interface associated with application (70). Again, such access may be granted to the service provider (60) after the application (70) has been installed, which may be achieved by downloading the application (70) from an application store. Each service provider (60) may also create an account using the application (70), as described above, and the account information may be stored in database (110) and capture information sufficient to enable each service provider (60) to be correctly identified.

FIG. 5 shows in greater detail segment 500 of FIG. 1 and, in particular, a consumer (30) using interface (280) generated by software application (40) to conduct a search for a listing of all allied health providers available for selection by the consumer (30). In this regard, the consumer (30) may be automatically presented with a listing of relevant search results, i.e. a listing of allied health service providers (60) who have minimum qualifications to provide the health-related service and satisfy one or more requirements sought by the consumer (30), and those who offer services in a geographical region (240) that includes the location (270) specified by the consumer (30). The one or more requirements specified by the user (30) may relate to, for example, details relating to the service that is being sought by the user (30), (eg. the cost of the service, a preference regarding whether the service is to be provided online or an in-person service, etc).

Accordingly, once the consumer enters relevant details relating to their location (residential/commercial address, current location, or preferred location), and subsequently enters a search query specifying the particular health-related service required by the consumer (30) and any additional requirements, the consumer (30) is presented with a listing of relevant search results representing a subset of all of the registered allied health service providers (60) present in database (110), with the results provided quickly and efficiently (substantially in real-time).

The consumer (30) may further filter search results according to additional search terms and other filtering criteria relating to any additional requirements of the user (30) (eg. a requirement that the service provider is male or female, has a particular accreditation, etc). In this regard, the application (40) may provide the consumer (30) with the ability to filter their searches according to a range of additional details relating to each service provider (60) including the services they offer and additional information from their profile such as details relating to their availability.

It is to be understood that in circumstances where it is not possible to clearly match a search query entered by a consumer (30) with information relating to a service provider (60) for the purpose of including the service provider (60) in search results (e.g. by way of an exact keyword match), the search functionality (140) may automatically include or exclude particular service providers (60) based upon a minimum similarity threshold analysis. In other words, provided a minimum similarity threshold is satisfied when the algorithm is used to generate search results by comparing the data presented by the consumer (30) in their search query with available data relating to available service providers (60), the service providers (60) will be included in the generated search results list. Where the similarity threshold is not satisfied, the particular service providers (60) will be excluded.

FIG. 5 also shows an example interface (290) which allows the consumer (30) to select a particular service provider (60) with a view to retrieving additional information relating to the particular service provider (60) and engaging with the service provider (60).

Interface (290) allows the consumer (30) to view additional details associated with the service provider (60) including the service provider's profile which may include information relating to costs associated with services provided by the service provider (60), additional characteristics associated with the service offerings, and the availability of the service provider (60). Of course, the consumer (30) may already be aware of some, or all, of the additional details since such details may already have been specified by the consumer (30) in their search request (eg. where the user request specified a particular cost, characteristic or availability of the service provider (60) as a requirement) or during subsequent filtering of search results. In any event, the full profile of a service provider may be displayed to the consumer (30). The software application (40) may enable the consumer (30) to control the type and/or extent of information that is displayed when the consumer views service provider information, including in profile views, search listings, etc.

Interface (310) is also shown in FIG. 5 as an example booking and payment interface, allowing consumers (30) to book and purchase services from a selected allied health service provider (60). The booking interface, which may display available date/time slots for appointments to be scheduled with the service provider (60), may be automatically updated in substantially real-time whenever changes to the service provider's schedule are made to ensure that all online bookings are captured and to prevent double bookings. The interface (310) also makes use of the payment gateway (170) to enable the consumer (30) to make payments (e.g. via credit card, EFT, PayPal and similar electronic payment platforms). Alternatively, if payment is not required in advance of the service, then such a payment interface (310) may be generated following the provision of services.

FIG. 6 shows in greater detail segment 600 of FIG. 1, and in particular, the provision of a particular health-related service (320) by a selected health service provider (60) to a consumer (30) (ie. after the consumer (30) has located the particular allied health service provider (60) utilising software application (40) and booked an appointment with the service provider (60)). In the particular embodiment shown, the service provider (60) provides a fitness training service and the particular exercise involves the consumer (30) running on a treadmill apparatus. However, the allied health services offered by service providers (60) are not limited to fitness training, and may relate to any allied health service including one or more of personal training, nutrition, weight loss and/or movement/mobility.

Following engagement of a service provider (60), but prior to the provision of services, the service provider (60) may publish material for viewing by the consumer (30), including for example one or more of instructional programs in order to prepare the consumer (30) for the particular fitness training (or other health-related service). Additional material may be published by service providers (60) including public materials viewable by all consumers (including potential customers), as well as a diary of the allied health provider (60) which may be viewable by consumers to enable the consumers to view available appointment times of the allied health service provider (60) as previously described.

It is to be further understood that whilst FIG. 6 illustrates an example of an in-person service delivery, the service or program may be delivered to the customer (30) by other means, including on-line or using a combination of both delivery methods.

FIG. 7 shows in greater detail segment 700 of FIG. 1 and, in particular, an embodiment in which payment may be effected to the service provider (60). In particular, the service provider (60) receives payments from clients (30) who have booked and paid on-line via software application (40) for their services, and the server (20) (using the payment gateway (170)) may automatically provide such payment to a bank account nominated by the service provider (330) (eg. a bank account associated with financial institution (80) of the service provider (60)). The payment made to the service provider (60) may represent a balance of a total payment made by the consumer (30) after a deduction is carried out in accordance with any processing and/or administrative fees.

As used herein, the term “server”, “system”, “computer”, “computing system” or the like may include any processor-based or microprocessor-based system including systems using microcontrollers, reduced instruction set computers (RISC), application specific integrated circuits (ASICs), logic circuits, and any other circuit or processor including hardware, software, or a combination thereof capable of executing the functions described herein. Such are exemplary only, and are thus not intended to limit in any way the definition and/or meaning of such terms.

The one or more processors as described herein are configured to execute a set of instructions that are stored in one or more data storage units or elements (such as one or more memories), in order to process data. For example, the one or more processors may include or be coupled to one or more memories. The data storage units may also store data or other information as desired or needed. The data storage units may be in the form of an information source or a physical memory element within a processing machine.

The set of instructions may include various commands that instruct the one or more processors to perform specific operations such as the methods and processes of the various embodiments of the subject matter described herein. The set of instructions may be in the form of a software program. The software may be in various forms such as system software or application software. Further, the software may be in the form of a collection of separate programs, a program subset within a larger program or a portion of a program. The software may also include modular programming in the form of object-oriented programming. The processing of input data by the processing machine may be in response to user commands, or in response to results of previous processing, or in response to a request made by another processing machine.

The diagrams of embodiments herein illustrate one or more control or processing units. It is to be understood that the processing or control units may represent circuits, circuitry, or portions thereof that may be implemented as hardware with associated instructions (eg, software stored on a tangible and non-transitory computer readable storage medium, such as a computer hard drive, ROM, RAM, or the like) that perform the operations described herein. The hardware may include state machine circuitry hardwired to perform the functions described herein. Optionally, the hardware may include electronic circuits that include and/or are connected to one or more logic-based devices, such as microprocessors, processors, controllers, or the like.

Optionally, the one or more processors may represent processing circuitry such as one or more of a field programmable gate array (FPGA), application specific integrated circuit (ASIC), microprocessor(s), and/or the like. The circuits in various embodiments may be configured to execute one or more algorithms to perform functions described herein. The one or more algorithms may include aspects of embodiments disclosed herein, whether or not expressly identified in the figures or a described method.

It will be appreciated by persons skilled in the relevant field of technology that numerous variations and or modifications may be made to the invention as detailed in the embodiments without departing from the spirit or scope of the invention as broadly described. The present embodiments are, therefore, to be considered in all aspects as illustrative and not restrictive.

Throughout this specification and claims which follow, unless the context requires otherwise, the word “comprise”, and variations such as “comprises” and “comprising”, will be understood to mean the inclusion of a stated feature or step, or group of features or steps, but not the exclusion of any other feature or step, or group of features or steps.

The reference to any prior art in this specification is not, and should not be taken as, an acknowledgement or any suggestion that the prior art forms part of the common general knowledge.

1. A computer-implemented method for facilitating the engagement of allied health advisors, the method comprising:
receiving, by one or more processors, data relating to a plurality of consumers seeking to engage one or more allied health service providers;
storing, by one or more processors, in a database including a data structure, data relating to a plurality of allied health service providers offering their services to consumers, the data comprising:
a geographical region in which the allied health service provider offers their services, and
one or more qualifications of the allied health service provider;
receiving, by one or more processors, a request from a consumer of the plurality of consumers that seek to identify, by interrogating the data structure, a subset of the plurality of the allied health service providers whose stored data satisfies the consumer request for an allied health service provider, the request including at least:
a health-related service sought by the consumer,
a location at which the consumer seeks to receive the health-related service, and
one or more requirements of the consumer in relation to the health-related service, including one or more of
a cost associated with the service,
a characteristic of the service, or
an availability of the consumer;
retrieving, by one or more processors, data relating to the consumer to which the request relates from the data relating to the plurality of consumers seeking to engage one or more allied health service providers;
generating and performing, by one or more processors, based upon the request received and the retrieved data relating to the consumer, a query of the data structure to identify a subset of the plurality of allied health service providers who offer services within a geographical region in which the location resides, have minimum qualifications required to provide the health-related service sought by the consumer, and satisfy the one or more additional requirements of the consumer, wherein the query comprises an exact keyword match query and a minimum similarly threshold analysis such that, when the exact keyword match query generates no relevant search results, the similarly threshold analysis is performed to generate results solely according to a comparison between the query and the data relating to the plurality of allied health service providers satisfying a minimum similarly threshold; and
providing, by one or more processors, to a data communications device associated with the consumer, a result of the search conducted according to the exact keyword match search query or a minimum similarity threshold analysis.

2. The computer-implemented method of claim 1, wherein the subset of allied health service providers included in the search query response are sorted according to one or more parameters selected by the consumer.

3. The computer-implemented method of claim 1, wherein the subset of allied health service providers included in the search query response are sorted automatically according to one or more pre-defined parameters.

4. A computer-implemented method according to claim 1, further comprising:
receiving, by one or more processors, from the data communications device associated with the consumer, a selection of a particular allied health service provider from the subset of allied health service providers.

5. The computer-implemented method of claim 1, wherein the stored data relating to the allied health service providers comprises details relating to the cost of services provided by the allied health service provider, the characteristics of services offered by the allied health service provider, and the availability of the allied health service provider, and when the one or more requirements specified in the user request specifies a particular cost of the service provider, characteristic of the service provider, and/or availability of the consumer, the method further comprises:
determining, by one or more processors, whether the one or more requirements are satisfied by comparing the one or more requirements against the stored data relating to the allied health service providers.

6. The computer-implemented method of claim 1, wherein the allied health service providers provide health-related services comprising one or more of:
personal training;
fitness training;
diet;
nutrition;
weight loss; or
movement/mobility.

7. The computer-implemented method of claim 1, wherein the one or more qualifications of the allied health service provider comprises one or more of:
an academic qualification;
a technical qualification;
experience;
an accreditation;
consumer rating; or
reputation.

8. The computer-implemented method of claim 1, wherein the data communications device associated with the particular consumer operates a software application that enables the particular consumer to prepare and submit the request, to view a response to the request, and to select a particular allied health service provider from the providers listed in the search query response.

9. The computer-implemented method of claim 8, wherein details relating to each allied health service provider are received via a data communications device that also operates a software application, the software application enabling each allied health service provider to submit details relating to the geographical region in which the allied health service provider offers their services, the qualifications of the allied health service provider, and additional details regarding the services they provide, prior to storage and indexing of same in the data structure.

10. The computer-implemented method of claim 8, wherein the software application further enables the allied health service providers to publish material for viewing by consumers, including public materials viewable by all consumers, and private materials viewable only by those consumers who have engaged the services of the allied health service provider.

11. The computer-implemented method of claim 10, wherein the published material comprises one or more of:
instructional programs created by the allied health service provider; or
a schedule of the allied health service provider.

12. The computer-implemented method of claim 11, wherein the published schedule of the allied health service provider is viewable by consumers using their data communications devices in a configuration enabling each consumer to view available appointment times of the allied health service provider and to schedule an appointment during one of the available times.

13. The computer-implemented method according of claim 12, wherein when an allied health service provider has been selected by the consumer and the engagement of the allied health service provider confirmed, the software application further provides a payment gateway enabling consumers to pay for services provided by the allied health provider comprising payment for access to instructional programs created by the service provider and the schedule of the service provider.

14. The computer-implemented method according of claim 13, wherein when an allied health service provider has been selected by the consumer and the engagement of the allied health service provider confirmed, the software application further provides a communication gateway enabling the consumer and allied health service provider to exchange messages and materials, engage in video conferencing, and/or schedule appointments to provide online or in-person services at an agreed time and location for an agreed cost.

15. The computer-implemented method of claim 8, wherein the software application further enables each of the consumers and the allied health service providers to monitor and update any submitted details, including any additional details submitted for the purpose of facilitating the identification of the consumer and/or allied health service provider.

16. A system for facilitating the engagement of allied health service providers, the system comprising one or more processors operable to:
receive data relating to a plurality of consumers seeking to engage one or more allied health service providers;
store, in a database including a data structure, data relating to a plurality of allied health service providers offering their services to consumers, the data comprising:
a geographical region in which the allied health service provider offers their services, and
one or more qualifications of the allied health service provider;
receive a request from a consumer of the plurality of consumers that seek to identify, by interrogating that data structure, a subset of the plurality of the allied health service providers whose stored data satisfied the consumer request for an allied health service provider, the request comprising at least:
a health-related service sought by the consumer,
a location at which the consumer seeks to receive the health-related service, and
one or more requirements of the consumer in relation to the health-related service including one or more of
a cost associated with the service,
a characteristic of the service, or
an availability of the consumer;
retrieve data relating to the consumer to which the request relates from the data relating to the plurality of consumers seeking to engage one or more allied health service providers;
generate and perform, based upon the request received and the retrieved data relating to the consumer, a query of the data structure to identify a subset of the plurality of allied health service providers who offer services within a geographical region in which the location resides have minimum qualifications required to provide the health-related service sought by the consumer, and satisfy the one or more additional requirements of the consumer wherein the query comprises an exact keyword match query and a minimum similarly threshold analysis such that, when the exact keyword match query generates no relevant search results, the similarly threshold analysis is performed to generate results solely according to a comparison between the query and the data relating to the plurality of allied health service providers satisfying a minimum similarly threshold; and
provide, to a data communications device associated with the consumer, a result of the search conducted according to the exact keyword match search query or the minimum similarly threshold analysis.

17. A non-transitory computer-readable medium having a plurality of computer instructions executable by one or more processors that, when executed, cause the one or more processors to:
receive data relating to a plurality of consumers seeking to engage one or more allied health service providers;
store, in a database including a data structure, data relating to a plurality of allied health service providers offering their services to consumers, the data comprising:
a geographical region in which the allied health service provider offers their services, and
one or more qualifications of the allied health service provider;
receive a request from a consumer of the plurality of consumers that seek to identify, by interrogating that data structure, a subset of the plurality of the allied health service providers whose stored data satisfied the consumer request for an allied health service provider, the request comprising at least:
a health-related service sought by the consumer,
a location at which the consumer seeks to receive the health-related service, and
one or more requirements of the consumer in relation to the health-related service including one or more of
a cost associated with the service,
a characteristic of the service, or
an availability of the consumer;
retrieve data relating to the consumer to which the request relates from the data relating to the plurality of consumers seeking to engage one or more allied health service providers;
generate and perform, based upon the request received and the retrieved data relating to the consumer, a query of the data structure to identify a subset of the plurality of allied health service providers who offer services within a geographical region in which the location resides have minimum qualifications required to provide the health-related service sought by the consumer, and satisfy the one or more additional requirements of the consumer wherein the query comprises an exact keyword match query and a minimum similarly threshold analysis such that, when the exact keyword match query generates no relevant search results, the similarly threshold analysis is performed to generate results solely according to a comparison between the query and the data relating to the plurality of allied health service providers satisfying a minimum similarly threshold; and
provide, to a data communications device associated with the consumer, the results of the search conducted according to the exact keyword match or the minimum similarly threshold analysis search query.
